(19) United States
(12) Patent Application Publication
(10) Pub. No.: US 2009/0291920 A1
(43) Pub. Date: Nov. 26, 2009

(54) DETECTION OF STEROID RECEPTORS ON CIRCULATING CARCINOMA CELLS AND TREATMENT

(75) Inventors: Ming Lu, Potomac, MD (US); Robert M. Lorence, Bethesda, MD (US)

Correspondence Address: Wellstat Management Company. LLC, LEGAL DEPARTMENT, 930 CLOPPER ROAD, GAITHERSBURG, MD, 20878, US

(73) Assignee: WELLSTAT BIOLOGICS CORPORATION, Gaithersburg, MD (US)

(21) Appl. No.: 12/296,458

(22) PCT Filed: Apr. 18, 2007
(86) PCT No.: PCT/US07/66849

§ 371 Date: Oct. 8, 2008

Related U.S. Application Data
(60) Provisional application No. 60/745,012, filed on Apr. 18, 2006.

(51) Int. Cl. A61K 31/566; C12Q 1/02; A61K 31/565; A61K 31/4353; A61K 31/4196

(52) U.S. Cl. 514/77; 435/29; 514/182; 514/300; 514/383

(57) ABSTRACT

The expression of a steroid receptor from circulating carcinoma cells in a blood sample is detected by isolating the carcinoma cells from the blood sample, making an extract from the isolated carcinoma cells and then performing on the extract a sensitive immunoassay capable of detecting the carcinoma cell-associated steroid receptor. A positive result indicates the presence of the steroid receptor in the carcinoma cells. This method can be used to identify cancer patients who are likely to benefit from treatment with an endocrine therapeutic agent.

### BACKGROUND OF THE INVENTION

Endocrine or hormonal therapy is one of the most important approaches in the treatment of breast and prostate cancer patients. Treatment is critically dependent upon the presence of steroid receptors in the tumors of these cancer patients. Steroid receptor status have been used for over 25 years in the decision making process for treating women with breast cancer. Endocrine or hormonal therapy is successfully used in the treatment of patients with estrogen receptor (ER) and progesterone receptor (PR) positive tumors (Bernard-Marty et al., 2004, The Oncologist 9:617-32). In addition, an important role of androgen receptor levels in tumors has also been found for response to endocrine therapy (Buchanan G et al., 2005, Cancer Res 65:8487-8496). Endocrine therapy results in a high rate of tumor responses (estimated to be 30% up to reports as high as 80% in selected patients, Gradishar, 2004, The Oncologist 9:378-84). Endocrine therapy of breast cancer has the advantage of combining high efficacy with minimal toxicity and a good quality of life (Bernard-Marty et al., 2004, The Oncologist 9:617-32).

There are two estrogen receptors: estrogen receptor-alpha and estrogen receptor-beta (Esslimani-Sahla et al., 2004, Clin Cancer Res 10:5769-5776. These two proteins exhibit a high degree of homology, especially in their DNA binding domains (Hanstein S et al., 2004, Eur J Endocrin 150:243-255; Matthews & Gustafsson J A, 2003, Mol. Interv. 3:181-92). The predictive value of ER-alpha level from tumor tissue is undisputed (Bardou et al., 2003, J Clin Oncol 10:1973-79). Besides estrogen receptors, the presence of progesterone receptors in breast cancer tissue has also been shown to provide an independent predictive factor for benefit from hormonal therapy (Bardou et al., 2003, J Clin Oncol 10:1973-79). Recently, tumor expression of estrogen receptor-beta and of androgen receptor has been shown to have an important role in the treatment of breast cancer patients (Hopp et al., 2004; Clin Cancer Res 10:7590-00; Buchanan et al, 2005, Cancer Res 65:8587-96).

Endocrine or hormonal therapy also plays an important role in the treatment of prostate cancer. Endocrine therapeutic agents against this cancer target the androgen receptor (AR) either indirectly via reducing the amount of circulating ligand or by directly inhibiting AR signaling (Scher et al., 2004, Endocrine-Related Cancer 11:459-76). Initially, AR is detecting in virtually all prostate cancers (Scher et al., 2004). However, after treatment, AR protein expression has been shown to be down-regulated in 30% of cases of hormone-refractory prostate cancer (Suzuki et al., 2003, Endocrine-Related Cancers 19:209-216).

The current clinical approaches for measuring steroid receptors use immunohistochemical (IHC) staining for the receptor from formalin-fixed biopsy material (Mann et al., 2005, J Clin Oncol 22:5148-54; Buchanan G et al., 2005, Cancer Res 65:8487-8496). Such approaches are time-consuming and often can give misleading results depending upon how the tissue was processed.

Specifically, significant rates of false negative results for ER expression have been reported using IHC. False negative rates of 20 to 30% are reported by D. Craig Allred, M.D. (www.breastcancerupdate.com/bcu2004/7/allred.htm) (Also see McCann, 2004 J Natl Cancer Inst 93:579-80 for general comments). False negative rates of at least 24% have been reported in the community setting. (Mann et al., 2005, J Clin Oncol 22:5148-5154, Discussion 2^{nd} paragraph).

This is a major clinical problem for women with breast cancer that is not being adequately addressed since hormonal therapy has such high efficacy and low toxicity. Furthermore, IHC is not practical for patients who do not have biopsy tissue available or inadequate samples for IHC.

Another problem that is not addressed by IHC testing of primary tumors is the issue of metastases developing a different receptor status from the primary tumor, especially going from receptor negative (from the primary) to receptor-positive (for the metastases). Getting a biopsy of the metastasis to perform IHC testing is often not a practical solution since this entails a surgical step. A preferred method would not entail any additional surgery for the patient. Eleven to 33% conversion rates from ER negative to positive have been reported:
11% of ER negative patients (based on results in their primary) subsequently had ER positive results for their metastases (Glass EL, 2003 et al., San Antonio Breast Cancer Symposium, Abstract #257)
11% of all patients tested (this would be 33% of the patients with ER-negativity based on assay of their primary tumor) subsequently had ER positive results for their metastases (Franco N, 2004, Proc ASCO, Abstract 539).
33% of 140 ER-negative patients (based on results in their primary) subsequently had ER positive results for their metastases (Spataro et al., 1992. Annals of Oncology 3:733-740, 1992)

Clearly, there is abundant evidence that many women who test estrogen or progesterone negative would benefit from hormonal therapies if a convenient assay were available either because of false-negative results of the IHC assay of their primary tumors or because their hormonal status changed in their metastases. Therefore, there is a need to better identify which patients express estrogen receptors (ER) and progesterone receptors (PR) and would benefit from therapy targeting such receptors. An assay of hormone receptors from circulating tumor cells in a blood sample would be most convenient for the patient. Unfortunately, previously used immunoassays of hormone receptors require at least 0.1 gram of tissue or approximately 0.1 ml of cytosol (Delage V et al., 1996 Clin Chem 42:1955-1960; Krambovitis et al., 1995 Clin Chem 41:48-53) which corresponds to approximately 1E+08 tumor cells [1 gram of tissue equals approximately 1 E+09 tumor cells (Cotran, Kumar and Robbins, 1989; Robbins Pathologic Basis of Disease, 4^{th} Edition, WB Saunders Co, Philadelphia, page 251, 2^{nd} column, 1^{st} paragraph, 2^{nd} sentence); Cytosol volume from 1 tumor cell equals approximately 1 picoliter (Kelemen et al., 2002, J Biol Chem 277:8741-48]. The reported immunoassay methods are markedly too insensitive to detect hormone receptors from very small numbers of circulating tumor cells (orders of magnitude less than 1E+08 per ml). This invention therefore provides a means for testing hormone receptors from circulating tumor cells and is therefore a convenient means to address the medical need of identifying additional patients for hormonal therapy.

### SUMMARY OF THE INVENTION

This invention provides a method of detecting the expression of a steroid receptor from circulating carcinoma cells in a blood sample comprising isolating the carcinoma cells from the blood sample, followed by making an extract (e.g. a lysate) from the isolated carcinoma cells followed by performing on the extract an immunoassay capable of detecting the steroid receptor, in which a positive immunoassay result indicates the presence of the steroid receptor in the carcinoma cells. In accordance with this invention the carcinoma cells are breast cancer cells and the steroid receptor is selected from the group consisting of estrogen receptor-alpha, estrogen receptor-beta, progesterone receptor and androgen receptor; or the carcinoma cells are prostate cancer cells and the steroid receptor is androgen receptor. The immunoassay is capable of detecting steroid receptor from nine hundred MCF-7 carcinoma cells which are spiked into a milliliter of a blood sample from a person without carcinoma.

This invention provides a method of identifying a cancer patient likely to benefit from treatment with an endocrine therapeutic agent, comprising the detection method described above. When used to identify such patients, the cancer cell-containing blood sample is drawn from the patient. This invention provides a method of treating cancer patients so-identified, which method comprises administering to the patient an endocrine therapeutic agent.

### BRIEF DESCRIPTION OF THE FIGURES

FIG. 1. ECL signal for immunoassay detection of recombinant ER-alpha using rabbit polyclonal antibodies Ab-5700 and Ab-31315 from Abcam (see Example 6 for details). Two conditions were tested using either TAG-31315 and BIOTIN-31315 or TAG-5700 and BIOTIN-31315 and using 1, 4, 16, 160 and 1600 pg/well of recombinant ER-alpha protein.

FIG. 2. ECL signal for the immunoassay detection of ER-alpha in lysates from MCF-7 breast cancer cells (positive control for ER-alpha expression). Shown is the data using lysates from 100, 500 and 900 cells per well.

FIG. 3. A comparison of the ECL signal for the immunoassay detection of ER-alpha in lysates from MCF-7 breast cancer cells (positive control for ER-alpha expression) versus K562 human leukemia cells (negative control). Data from the same experiment as shown in FIG. 2 is used for this figure, except that the following additional data is used: data obtained from lysate material from 10,000 MCF-7 cells; and data obtained from MCF-7 cells (lysates from 100, 500, 900, and 10,000 cells per well).

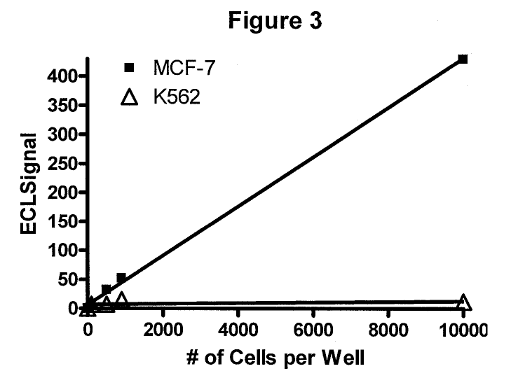

### DETAILED DESCRIPTION OF THE INVENTION

As used herein the transitional term “comprising” is open-ended. A claim utilizing this term can contain elements in addition to those recited in such claim. Thus, for example, the claims can read on methods that also include other steps not specifically recited therein, as long as the recited elements or their equivalent are present.

The following abbreviations are used herein:

AR: androgen receptor
ER: estrogen receptor
PR: progesterone receptor
IHC: immunohistochemistry
ECL: electrochemiluminescence

This invention provides methods sensitive enough for quantifying the levels of steroid receptor protein (estrogen receptor-alpha, estrogen receptor-beta, progesterone receptor, and/or androgen receptor) in circulating breast or prostate cancer in blood samples and provides methods for identifying those patients who are likely to benefit from therapy using hormonal therapy or another agent targeted to steroid receptors such as estrogen receptors and/or progesterone receptors. Thus this invention advances the breast and prostate cancer treatment fields by providing a convenient, highly sensitive and rapid means to test blood samples to identify additional patients who would benefit from hormonal therapy. A rapid and highly sensitive immunological assay to detect estrogen receptor-alpha, estrogen receptor-beta, androgen receptor, and/or progesterone receptor proteins in circulating breast cancer cells or to detect androgen receptor proteins in circulating prostate cancer cells, such as using electrochemiluminescence (ECL)-detection, is a preferred means to identify additional patients who would benefit from hormonal therapy.

This invention is based on combining the high specificity of procedures used to isolate circulating carcinoma cells from blood with the high sensitivity of certain immunologically based assays such as ECL. Circulating breast or prostate cancer cells are first enriched using immunomagnetic beads by isolating and purifying the circulating cancer cells from blood.

In an embodiment of the detection method of this invention the sensitivity level of the immunological assay is such that the assay is capable of detecting steroid receptors (i.e., estrogen receptor-alpha, estrogen receptor-beta, progesterone receptor, or androgen receptor from carcinoma cells in blood at a concentration of between thirty and 900 carcinoma cells per milliliter of blood, preferably between thirty and 100 carcinoma cells per milliliter of blood, more preferably between ten and thirty carcinoma cells per milliliter of blood, more preferably between three and ten carcinoma cells per milliliter of blood, and most preferably between one and three carcinoma cells per milliliter of blood. In a more specific embodiment the carcinoma cells are MCF-7 breast cancer cells. MCF-7 cells are known to express estrogen receptor-alpha (Detre S et al., 2003, Cancer Res 63:6516-22), estrogen receptor-beta (Hopp T A et al., Clin Cancer Res 10:7490-9), progresterone receptor (Detre et al., 2003), and androgen receptor (Buchanan G et al., 2005, Cancer Res 65:8487-96).

A preferred means to isolate carcinoma cells and test such samples for hormone receptors is as follows:

A sample (usually in the range of approximately 8 to 20 ml) of blood from a patient with cancer, especially breast cancer, is taken. Steps include as detailed below:
1. Removal of red blood cells
2. Optional negative selection to further deplete normal leukocytes. A preferred embodiment includes this step.
3. Positive selection for circulating carcinoma cells
4. Extraction of nuclear proteins including estrogen receptors and/or progesterone receptors
5. Detection and quantification of estrogen receptors and/or progesterone receptors from circulating carcinoma cells

### 1. Removal of Red Blood Cells.

A variety of methods are available to remove red cells including but not limited to separation based on density (such as collection of blood directly into the Becton Dickinson BD Vacutainer CPT tubes) followed by centrifugation) and commercial lysing buffers such as PURESCRIPT RBC lysis buffer (Gentra, Minneapolis), FACS lysing solution (BDIS), IMMUNOLYSE (Coulter), OPTILYSE B (Immunotech), and ACK lysing buffer (Biosource, Rockville, Md.).

A preferred method uses the BD Vacutainer CPT tubes with anticoagulant (EDTA or citrate). These tubes contain a material that upon correct centrifugation (1,100×g for 10 minutes, swing-out bucket rotor) allows for elimination of red blood cells and neutrophils. After centrifugation, the bottom of the tube contains a cell pellet of erythrocytes (red blood cells) and neutrophils. Above the cell pellet is a gel barrier and above the gel barrier are tumor cells, lymphocytes and monocytes as a band at the bottom of the plasma. The tumor cells, lymphocytes and monocytes can then be readily collected from the top above the gel barrier. This method is preferred as it removes not only the red blood cells but also the neutrophils.

### 2. Negative Selection to Further Deplete Normal Leukocytes.

A preferred embodiment of this invention uses of negative selection step for isolation of tumor cells. Negative selection allows for further depletion of leukocytes especially the lymphocytes and monocytes. This step comprises the use of antibodies that are bispecific for both leukocyte antigens, especially CD45, the common leukocyte antigen, and for a red blood cell antigen such as glycophorin A. A commercially available cocktail of such bispecific antibodies is available from Stemcell Technologies (Rosettesep Catalog #15127 and #15167). This cocktail includes bispecific antibodies against glycophorin A and against a variety of cell surface antigens on human hematopoietic cells (CD2, CD16, CD19, CD36, CD38, CD45, CD66b). One or more of these bispecific antibodies are added to the BD Vacutainer CPT tubes before blood collection. In a preferred embodiment, the cocktail of bispecific antibodies against more than one leukocyte-associated CD molecule is used. When the blood is introduced into the CPT vacutainer tube, the bispecific antibodies form immunorosettes each consisting of leukocytes plus many red blood cells. These immunorosettes have a density approximately that of red blood cells and when centrifuged are found in the red blood cell pellet, thus further removing leukocytes from the tumor cell fraction found above the cell pellet and gel barrier. The fraction with the tumor cells in plasma is collected for further processing.

### 3. Positive Selection for Circulating Carcinoma Cells.

A preferred method of isolating circulating carcinoma cells uses immunomagnetic beads. Other methods of isolation of circulating cancer cells include filtration (Vona G et al., 2000, Am J Pathol. 2000 156:57-63). In a preferred embodiment, the immunomagnetic beads have antibodies against antigens found selectively on the surface of carcinoma cells such as epithelial cell adhesion molecule (EpCAM), cytokeratins such as cytokeratin-19 and especially a cocktail of antibodies against cytokeratins and other surface markers. The immunomagnetic beads may be of various sizes (50 microns to less than 200 nm) and include DYNAL beads (>1.5 microns to about 50 microns) with antibodies against EpCAM (which are commercially available). In an embodiment of the invention, EASYSEP™ human EpCAM positive selection cocktail and EASYSEP™ magnetic nanoparticles (Stemcell Technologies) are added to the fraction with the tumor cells in plasma from the previous step. A magnet is then used to separate tumor cells from the rest of the material and the tumor cells are washed with an aqueous solution.

### 4. Extraction of Nuclear Proteins Including Steroid Receptors.

In the next step, enriched or purified tumor cells are then ready for extraction of nuclear proteins. A variety of commercially available kits can be used such as:
Sigma CelLytic™ NuClEAR™ Extraction Kit (Product Code: NXTRACT; Sigma, St. Louis, Mo. 63103)
Pierce NE-PER Nuclear and Cytoplasmic Extraction Reagents Kit (Product Number 78833; Pierce Biotechnology, Inc., Rockford, Ill. 61105)
Nuclear extraction kit (Cat #AY2002, Panomics, Inc., Redwood City, Calif. 94063)

A preferred extraction method uses RIPA buffer (Pierce Biotechnology, Catalog #89900) along with 0.4 M to 0.45 M potassium chloride (KCl).

5. Detection and Quantification of Steroid Receptors from Circulating Carcinoma Cells.

Detection of estrogen receptor-alpha, progesterone receptor, or androgen receptors can then be accomplished by use of a highly sensitive sandwich immunoassay using antibodies which bind to the receptor being assayed. A variety of antibodies can be used for the immunoassay, preferably using at least one polyclonal antibody and most preferably, using two polyclonal antibodies. In an embodiment of this invention the immunoassay uses a first polyclonal antibody against the N-terminal region of the steroid receptor and a second polyclonal antibody against the C-terminal region of the steroid receptor.

Detection of the steroid receptor can be accomplished by using a sandwich immunoassay with the two sets of antibodies directed against the steroid receptor. In a preferred embodiment using electrochemiluminescence, one antibody is linked to biotin and the other linked to a ruthenium detecting molecule.

In a preferred embodiment of the invention, an antibody against the steroid receptor is linked with biotin and a second antibody against the steroid receptor is labeled with a detecting molecule. In the case of electrochemiluminescence (ECL), the detecting molecule is ruthenium. There is abundant literature in the public domain that provides amply useful methods for linking ruthenium to antibodies (e.g., Lee et al., Am J Trop Med Hyg 2001, 65:1-9). The nuclear extract is mixed with the two antibodies and incubated briefly followed by the addition of streptavidin-coated magnetic beads in a solution containing tripropylamine. With application of an electric potential and in the presence of the target antigen (ER), the ruthenium label is excited and light is emitted and detected using an ECL detecting instrument (such as the ORIGEN analyzer or a commercially available instrument like the M-Series® 384 from BioVeris Corporation, Gaithersburg, Md.)

In a preferred embodiment of the invention, the immunoassay utilized in accordance with this invention can use one of the following combinations:
1. Two sets of polyclonal antibodies against ER-alpha (the most preferred embodiment)
2. A polyclonal antibody and a monoclonal antibody against ER-alpha.
3. Two monoclonal antibodies against ER-alpha.

For purposes of detection of ER-alpha, a variety of monoclonal and polyclonal antibodies against ER-alpha are useful and include, but are not limited to the following antibodies against ER-alpha:
Polyclonal antibody against ER-alpha (e.g., Rabbit anti-ER-alpha polyclonal antibodies from BETHYL Laboratories (Montgomery, Tex.): Catalog Numbers #A300-495A, A300-496A, A300-497A; and A300-498A); Catalog #29595, AnaSpec, San Jose, Calif. 95131; Abcam Catalog Numbers ab3575; ab5700-1; ab30656; ab28798; ab28799; ab31312; ab31314-5; 31478; Abcam, Inc., Cambridge, Mass. 02139; LabVision Catalog Number RB-9061, LabVision, Fremont, Calif. 94539; LabVision Catalog Number RB-9451, LabVision, Fremont, Calif. 94539)
Monoclonal antibodies against ER-alpha (LabVision Catalog Numbers: MS-750; MS-354; MS-391; MS-1071; RM-9101; RT-1639; RT-1640; RT-1641; RT-1642; Abcam catalog numbers: ab31478; ab1104; ab858; ab484; ab2746; ab9269; ab8857; ab7820-8; ab19348-9; ab15284)

The immunoassay of this invention is more rapid and has a significantly greater sensitivity than any previously developed immunoassay for steroid receptors. The immunoassay of this invention is capable of detecting steroid receptor expression from 900 MCF-7 breast cancer cells added per ml of blood from a human volunteer without cancer, preferably 500 MCF-7 cells per ml of blood, more preferably 100 MCF-7 cells per ml of blood, and most preferably 30 MCF-7 cells per ml of blood.

Besides electrochemiluminescence, other immunoassays that can yield a high sensitivity required for this application include, but are not limited to:
a) Chemiluminescence such as described by Liu Y et al., 2003 (J Food Protection 66:512-7).
b) Fluorogenic-chemiluminescence (FCL) as described by Yu H et al., 2000 (Biosens Bioelectron 14:829-40)
c) Fluoresence polarization immunoassay (see Howanitz J H, 1988 Arch Pathol Lab Med 112:775-9)
d) Time-resolved fluorescence immunoassay (Butcher H et al., 2003, J Immunol Methods 272:247-56; Soukka et al., 2001, Clin Chem 47:1269-78; Howanitz J H, 1988 Arch Pathol Lab Med 112:775-9)

In a preferred embodiment, the relative quantity of breast cancer cells used in the assay is estimated. This allows for a ratio of total ER-alpha protein per cell to be obtained and can be compared to control standards of breast cancer cells with high, moderate, and low levels of ER-alpha protein per cell. This is a preferred embodiment since it eliminates a false positive situation in which there are many circulating breast cancer cells that have a low level of ER-alpha protein expression that may give a signal that mimics that obtained from a small number of breast cancer cells with a high level expression. In this embodiment, a variety of approaches can be used to estimate relative cell numbers including flow cytometic analysis, quantification of total DNA or DNA related antigens such as histones from lysed cells (there is 6 pg DNA per diploid cell), quantification of a house-keeping gene product like beta-actin, and turbidity or absorbance measurements.

Due to its sensitivity the method according to this invention for identifying breast cancer patients likely to benefit from treatment with a hormonal agent can be fruitfully applied to patients from whom a tumor biopsy tissue had been previously determined (e.g. by immunohistochemistry) to be negative for ER expression by a tissue assay for ER.

An assay for ER-beta can be performed using the same methods as for ER-alpha, except that antibodies specific toward ER-beta are used. Examples of useful antibodies against ER-beta include, but are not limited to:
Rabbit polyclonal antibody against ER-beta (e.g., Catalog Number Erb15-A, Alpha Diagnostics International, Inc., San Antonio, Tex.; Catalog Number ab5786, Novus Biologicals, Littleton, Colo.)
Mouse monoclonal antibody against ER-beta (e.g., Catalog Number ab16813, Novus Biologicals)

An assay for PR can be performed using the same methods as for ER-alpha, except that antibodies specific toward PR are used. Examples of useful antibodies against PR include, but are not limited to:
Rabbit polyclonal antibody against PR (e.g., Abcam Catalog Numbers ab15509; ab 15510; Abcam, Inc., Cambridge, Mass. 02139)
Mouse monoclonal antibodies against PR (e.g., Abcam Catalog Numbers ab9895; ab9896; ab2764; Abcam, Inc.)
Rabbit monoclonal antibody against PR (e.g., Abcam Catalog Number ab27616)

An assay for androgen receptor (AR) can be performed using the same methods as for ER, except that antibodies specific toward AR are used. Examples of useful antibodies against PR include, but are not limited to:
Goat polyclonal antibody against AR (e.g., Abcam Catalog Number ab19066; Abcam, Inc., Cambridge, Mass. 02139)
Rabbit polyclonal antibody against AR (e.g., Abcam Catalog Number ab3509)

Hormonal therapeutic agents (also called endocrine therapeutic agents) for the treatment of breast cancer in this invention include, but are not limited, to those approved by the US Food and Drug Administration include (Buzdar, 2003, The Oncologist 8:335-41):
Tamoxifen (see also Gradishar, 2004, The Oncologist 9:378-84)
Aromatase inhibitors (anastrozole, letrozole, exemestane; see also Campos 2004 (The Oncologist 9:126-36)
Fulvestrant, the estrogen receptor antagonist that downregulates estrogen and progresterone receptors and has no known agonist activity (see also Bross et al., 2002, The Oncologist 7:477-80)

The aromatase inhibitor, fadrozole, has been approved in Japan.

The invention will be better understood by reference to the following examples, which illustrate but do not limit the invention described herein.

### EXAMPLES

### Example 1

A patient with breast cancer comes into the office and a blood sample is collected in a tube to prevent clotting. Cancer cells are isolated and the nuclear proteins extracted using a commercially available kit such as Sigma CelLytic™ NuClEAR™ Extraction Kit. A ruthenium-labeled rabbit polyclonal antibody against ER-alpha and a biotinylated polyclonal antibody (also against ER-alpha) are added and the followed by the addition of a suspension of magnetic beads with strepavidin attached and then a solution containing tripropylamine. An electric current is applied and electrochemiluminescence (ECL) is detected using an ECL detection device such as one commercially available (BioVeris Corporation). The signal is proportional to the amount of ER-alpha receptor found in the circulating tumor cells.

### Example 2

A patient with an elevated level of ER-alpha on circulating malignant cells as indicated in Example 1 is then treated with a hormonal therapy.

### Example 3

Methods are as in example 1, except the antibodies are against progesterone receptor (PR)

### Example 4

A patient with an elevated level of ER-alpha or PR on circulating malignant cells as indicated in Examples 1, 2, or 3 is then treated with hormonal therapy

### Example 5

In this example, the sensitivity of detecting ER-alpha from breast cancer cells using a sandwich immunoassay using electrochemiluminescence is examined.

A PBS assay buffer is prepared:
Assay Buffer: 0.5% Tween-20 and 0.5% bovine serum albumin (BSA) in PBS (phosphate buffered saline)

Rabbit anti-ER-alpha polyclonal antibody (preferably this polyclonal antibody is against the full-length ER-alpha protein) is first obtained in both biotinylated and non-biotinylated forms. The non-biotinylated polyclonal antibody is ruthenium labeled (“TAG-labeled”) as follows:
1.5 μg/μl ruthenium label (BV-TAG-NHS Ester, Catalog #110034; BioVeris Corporation, Gaithersburg, Md., USA) is prepared in DMSO.
For 500 μl of antibody, 18.8 μl BV-TAG-NHS is added and for 200 μl of polyclonal antibody, 3.8 μl BV-TAG-NHS is added. In each case, the solution is incubated for one hour and the reaction stopped by the addition of 20 μl of 2M glycine.
Uncoupled BV-TAG-NHS Ester in each reaction mixture is removed using a PD-10 gel filtration column, pre-equilibrated with PBS (including 0.08% sodium azide), which is also used for elution. For each labeled antibody, the protein concentration in each fraction is determined by protein assay and the fraction with high protein content is used in subsequent examples.

The ruthenium-labeled polyclonal antibody and the biotinylated polyclonal antibody are referred hereafter in this example as “TAG-pAb” and “Biotin-pAb”.

MCF-7 breast cancer cells (from ATCC, Manassas, Va.) are grown in 6-well tissue culture plates as per ATCC recommended conditions, washed two times with PBS, and an aliquot counted using a hemacytometer. Extraction of nuclear proteins from MCF-7 cells is performed using the Sigma CelLytic™ NuClEAR™ Extraction Kit as per manufacturer's recommendation.

An electrochemiluminescence assay is performed as follows:
Sequentially, to each well, extracts from MCF-7 cells are added (the amount of extract per well is varied from that extracted from 30 to 100 MCF-7 cell; control wells without extract are also used) and then 50 μl/well of a mixture of TAG-Ab and Biotin-Ab (e.g., at a concentration between 0.5 to 2 μg/ml each; diluted into the PBS assay buffer) are added to wells of a 96-well U-bottom polypropylene plate and are incubated at room temperature with constant shaking (e.g., for 2 hours).
10 μg of magnetic streptavidin beads (e.g., Dynabeads M-280 Streptavidin, Catalog #110028, BioVeris, Corporation, Gaithersburg, Md.) in 25 μl is added to each well and incubated with constant shaking (e.g., for 30 minutes).
PBS assay buffer is added to each well to make a final volume of 250 μl per well. All conditions are tested in at least duplicate wells. The 96 well plate is then analyzed for electrochemiluminescence using the M8 M-Series® Analyzer (Catalog Number 310800, BioVeris, Corporation, Gaithersburg, Md.).

Using this immunoassay, ER-alpha is detectable and above baseline from extracts from at least 100 MCF-7 cells per well.

### Example 6

In this example, the sensitivity of detecting recombinant human estrogen receptor (ER)-alpha was examined using a sandwich immunoassay using electrochemiluminescence.

A PBS assay buffer was prepared:
Assay Buffer: 0.5% Tween-20 and 0.5% bovine serum albumin (BSA) in PBS (phosphate buffered saline, pH 7.2)

Two sets of anti-ER-alpha rabbit polyclonal antibody were first obtained from Abcam Inc (Cambridge, Mass.):
Ab-5700 which is directed to the C-terminus; and
Ab-31315 which is directed to the non-phosphorylated site of amino acid Serine 167 of human estrogen receptor-alpha.

A portion of both polyclonal antibodies was ruthenium labeled (“TAG-labeled”) and a portion of Ab-31315 was biotin-labeled of according the methods of Lorence & Lu (WO 2006/041959 A2). The ruthenium-labeled polyclonal antibody Ab5700 and the ruthenium-labeled polyclonal antibody Ab31315 are hereafter in referred to as “TAG-5700” and “TAG-31315” respectively. Biotinylated polyclonal antibody Ab31315 is referred hereafter in this example as “BIOTIN-31315”.

Recombinant estrogen receptor alpha protein was obtained from Invitrogen (Carslbad, Calif.; Catalog number #P2187, 2600 pmol/ml).

An electrochemiluminescence assay was performed as follows:
Standards were diluted in Assay Buffer to yield 1600, 160, 16, 4 and 1 pg/well when 25 μL was used per well. To each well of a 96-well U-bottom polypropylene plate (with 25 μL of standard per well) were added 50 μl/well of a mixture of either:
   TAG-31315 and BIOTIN-31315 (e.g., at a concentration of 1.0 μg/ml in the 50 μl prior to addition); or
   TAG-5700 and BIOTIN-31315 (e.g., at a concentration of 1.0 μg/ml in the 50 μl prior to addition)
The resultant solution was incubated at room temperature with constant shaking (for 2 hours).
10 μg of magnetic streptavidin beads (e.g., DYNABEADS M-280 Streptavidin, Catalog #110028, BioVeris Corporation, Gaithersburg, Md.) in 25 μl was added to each well and incubated with constant shaking (for 30 minutes).
PBS Assay Buffer was added to each well to make a final volume of 250 μl per well. All conditions were tested in duplicate wells. The 96 well plate was then analyzed for electrochemiluminescence using the M-Series® 384 Analyzer (BioVeris Corporation, Gaithersburg, Md.).

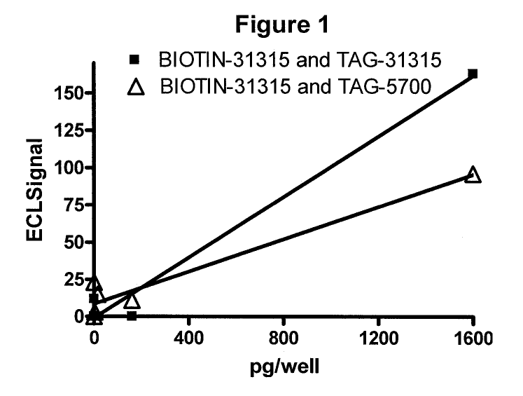

Using this immunoassay, 1600 pg per well of ER standard was detectable with a signal above background (FIG. 1).

### Example 7

In these sets of experiments, initially a repeat determination of the sensitivity of detecting recombinant ER-alpha using the same antibodies as in Example 6 was performed. However, storage of these antibodies at 4° C. led to a loss in sensitivity with the ECL signal for 1250 pg per well being below baseline and the ECL signal from 12500 pg per well being 160 ECL units above baseline. With this loss in sensitivity, additional antibodies were then tested. Chicken polyclonal IgY antibody against ER-alpha was obtained from GenWay Biotech, Inc. (San Diego, Calif.; catalog #15-288-21182). A portion of this polyclonal antibody was ruthenium labeled (“TAG-IgY”) and a portion was biotin-labeled (“BIOTIN-IgY” of according the methods of Lorence & Lu (WO 2006/041959 A2).

An ECL immunoassay using ER-alpha standards was performed with these antibodies according to the methods of Example 6. Using these IgY antibodies, the lowest amount tested (12.5 pg per well of ER-alpha) was detectable and gave an ECL signal above baseline (Table 1).

TABLE 1
Electrochemiluminescence (ECL) detection of recombinant ER-alpha
by immunoassay using ruthenium-labeled chicken polyclonal (TAG-IgY)
and biotinylated chicken polyclonal antibody (Biotin-IgY).

| ER-alpha (pg/well) | Mean ECL Signal (above background)* |
|---|---|
| 12.5 | 285 |
| 125 | 819 |
| 1250 | 3735 |
| 12500 | 8551 |
| 125,000 | 10,101 |

*Mean ECL signal above the mean signal from control wells with no antigen.

Using these IgY antibodies, cell extracts from MCF-7 human breast carcinoma cells (positive control cells for ER-alpha expression) were analyzed.

MCF-7 cells (from ATCC, Manassas, Va.) were grown in tissue culture as per ATCC recommended conditions, washed two times with PBS, and an aliquot counted using a hemacytometer. Lysis of SK-BR-3 cells and obtaining the supernatant was performed using the Pierce Lysis Buffer [M-PER catalog #78501; Pierce Biotechnology, Rockford, Ill.] with Pierce protease inhibitor [catalog #78410; Pierce Biotechnology]. The amount of lysate supernatant per well was varied from that extracted from 8 to 125,000 MCF-7 cells and analyzed for ER using the immunoassay with IgY antibodies. Since little if any ECL signal was seen using this extraction with M-PER, this was attributed to poor extraction efficiency.

A variety of extraction methods were next used. Tested were the use of:
RIPA extraction buffer (Pierce Biotechnology, Catalog #89900)
RIPA buffer plus 0.4M potassium chloride (KCl)
RIPA buffer plus heating at 95° C. for 2 minutes
RIPA buffer plus shearing with a spin column (QIAGEN)
Nuclear extraction kit from Marligen (Cat #11906-100)

RIPA buffer with KCl was found to be the best of all of these approaches when testing MCF-7 cells (Table 2).

TABLE 2
Electrochemiluminescence (ECL) detection of ER-alpha by ECL
immunoassay using ruthenium-labeled chicken polyclonal (TAG-IgY)
and biotinylated chicken polyclonal antibody (Biotin-IgY)
and using RIPA-KCL extraction of MCF-7 cells.

| MCF-7 cells/well | Mean ECL Signal (above background)* |
|---|---|
| 500 | 69 |
| 10,000 | 210 |

*Mean ECL signal above the mean signal from control wells with no antigen.

In parallel to this assay using MCF-7 cell extracts, another standard curve was performed and a decrease in sensitivity was noted (compare Table 3 with Table 1). For this reason, in the next example, additional storage conditions for IgY were examined in order to improve antibody stability. In addition, additional antibodies were also examined.

TABLE 3
Repeat of electrochemiluminescence (ECL) detection of recombinant
ER-alpha by immunoassay using ruthenium-labeled chicken polyclonal
(TAG-IgY) and biotinylated chicken polyclonal antibody (Biotin-IgY).

| ER-alpha (pg/well) | Mean ECL Signal (above background)* |
|---|---|
| 4 | 126 |
| 16 | 2** |
| 160 | 293 |
| 1600 | 1982 |

*Mean ECL signal above the mean signal from control wells with no antigen.
**These low ECL values were most consistent with a temporary problem with the ECL detection instrument during the analysis of these wells.

### Example 8

In this example, a sandwich immunoassay using electrochemiluminescence was used to examine:
the sensitivity of detecting recombinant ER-alpha
the sensitivity of detecting ER-alpha from MCF-7 breast cancer cells
the specificity of detecting ER-alpha from MCF-7 (positive control cells for high expression of ER-alpha) vs. K562 leukemia cells (negative control cells)

A new shipment of chicken anti-ER-alpha IgY polyclonal antibody was obtained from GenWay, Catalog Number #15-288-21182. A portion of this antibody stock was again biotinylated and a portion was again labeled with ruthenium according to the methods of Lorence & Lu (WO 2006/041959 A2). The ruthenium-labeled chicken polyclonal antibody and the biotinylated chicken polyclonal antibody are referred hereafter in this example as “TAG-IgY2” and “BIOTIN-IgY2”. Following labeling, these had concentrations of 201 μg/mL and 124 μg/mL, respectively. Following labeling, a portion of TAG-IgY2” and “BIOTIN-IgY2” was each stored at 4° C. without the addition of any BSA and a portion of TAG-IgY2” and “BIOTIN-IgY2” was each stored at 4° C. with the addition of 1% BSA.

Four different rabbit anti-ER-alpha polyclonal antibodies were obtained from BETHYL Laboratories (Montgomery, Tex.):
Catalog Number #A300-495A: Polyclonal antibody against the epitope of ER-alpha which maps to a region of the protein between amino acid residues 1 and 50 and is therefore directed to the N-terminus. This antibody was labeled with ruthenium according to the methods of Lorence & Lu (WO 2006/041959 A2). It is referred to hereafter in this example as “TAG-495A”. Following labeling, it had a concentration of 483 μg/mL and was then stored at 4° C. without the addition of any BSA.
Catalog Number #A300-496A: Polyclonal antibody against the epitope of ER-alpha which maps to a region of the protein between amino acid residues 100 and 150 and is therefore directed to a region near the N-terminus. This antibody was labeled with biotin according to the methods of Lorence & Lu WO 2006/041959 A2). It is referred to hereafter in this example as “BIOTIN-496A”. Following labeling, it had a concentration of 486 μg/mL and was then stored at 4° C. without the addition of any BSA.
Catalog Number #A300-497A: Polyclonal antibody against the epitope of ER-alpha which maps to a region of the protein between amino acid residues 525 and 575 and is therefore directed to a region closer to the C-terminus than antibodies A300-495A and A300-496A. This antibody was labeled with ruthenium according to the methods of Lorence & Lu (WO 2006/041959 A2). It is referred to hereafter in this example as “TAG-497A”. Following labeling, it had a concentration of 622 μg/mL and was then stored at 4° C. without the addition of any BSA.
Catalog Number #A300-498A: Polyclonal antibody against the epitope of ER-alpha which maps to a region of the protein between amino acid residues 550 and the C-terminus. This antibody was labeled with biotin according to the methods of Lorence & Lu (WO 2006/041959 A2). It is referred to hereafter in this example as “BIOTIN-498A”. Following labeling, it had a concentration of 335 μg/mL and was then stored at 4° C. without the addition of any BSA.

Recombinant human ER-alpha (from Invitrogen) was the same as used in example 6.

An electrochemiluminescence assay was performed as follows:
Standards were again diluted in Assay Buffer to yield 1600, 160, 16, and 4 pg/well when 25 μL was used per well. To each well of a 96-well U-bottom polypropylene plate (with 25 μL of standard per well) were added 50 μl/well of a mixture of a TAG antibody and a biotinylated antibody (at a concentration of 1.0 μg/ml in the 50 μl prior to addition) and the resultant solution was incubated at room temperature with constant shaking (for 2 hours). In this assay, four sets of antibody conditions were tested:
   CONDITION #1: TAG-IgY2” and “BIOTIN-IgY2” stored at 4° C. without the addition of any BSA following labeling.
   CONDITION #2: TAG-IgY2” and “BIOTIN-IgY2” stored at 4° C. with the addition of 1% BSA to each labeled antibody after labeling occurred.
   CONDITION #3: TAG-495A” and “BIOTIN-498A” (This pair of antibodies was selected as these were antibodies against epitopes from two different regions of the protein.)
   CONDITION #4: TAG-497A” and “BIOTIN-496A” (This pair of antibodies was selected as these were antibodies against epitopes from two different regions of the protein.)
10 μg of magnetic streptavidin beads (e.g., Dynabeads M-280 Streptavidin, Catalog #110028, BioVeris Corporation, Gaithersburg, Md.) in 25 μl was added to each well and incubated with constant shaking (for 30 minutes).
PBS Assay Buffer was added to each well to make a final volume of 250 μl per well. All conditions were tested in at least duplicate wells. The 96 well plate was then analyzed for electrochemiluminescence using the M-Series® 384 Analyzer (BioVeris Corporation, Gaithersburg, Md.).

Using CONDITIONS #2, #3, and #4, as little as 4 pg per well of ER-alpha standard had a mean ECL signal above background (Table 4). Using CONDITION #1, as little as 16 pg per well of ER-alpha standard was detectable with a signal above background (Table 4).

CONDITION #2 in which the labeled IgY antibodies (which were at lower concentrations than any of the 4 rabbit antibodies) were stored in 1% BSA gave advantageous results compared to CONDITION #1 in which the labeled IgY was not stored with the addition of BSA.

CONDITIONS #3 and #4 using the rabbit polyclonal antibodies gave advantageous results compared to CONDITIONS #1 and #2 using the chicken polyclonal antibody. Furthermore, CONDITION #3 using BIOTIN-498A and TAG-495A gave advantageous results compared to CONDITION #4 using BIOTIN-496A and TAG-497A.

TABLE 4
Electrochemiluminescence (ECL) detection of recombinant
ER-alpha by immunoassay using four different antibody
conditions (see text of this Example)

Mean ECL Signal (above background)

| ER-alpha | CONDI- | CONDI- | CONDI- | CONDI- |
|---|---|---|---|---|
| (pg/well) | TION #1 | TION #2 | TION #3 | TION #4 |
| 4 | Negative* | 19 | 24 | 6 |
| 16 | 25 | 28 | 101 | 12 |
| 160 | 87 | 77 | 983 | 132 |
| 1600 | 596 | 762 | 9592 | 1217 |

*Negative: Mean ECL signal below background level.

### Example 9

In this example, a sandwich immunoassay using electrochemiluminescence was used to examine:
the sensitivity of detecting ER-alpha from MCF-7 breast cancer cells
the specificity of detecting ER-alpha from MCF-7 (positive control cells for high expression of ER-alpha) vs. K562 leukemia cells (negative control for ER-alpha)

Procedures were as in EXAMPLE 8 using CONDITION #3 (BIOTIN-498A and TAG-495A) with the following additional steps of obtaining cell extracts:
MCF-7 breast cancer cells and K562 leukemia cells (from ATCC, Manassas, Va.) were grown as per ATCC recommended conditions, were washed two times with PBS, and an aliquot counted using a hemacytometer. Extraction of ER-alpha from each cell line (5 million cells per mL of extraction buffer) was performed using a modified RIPA buffer (Pierce Catalog #89900) with the addition of 0.45M potassium chloride (KCl) and 5 mM ethylendiaminetetraacetic acid (EDTA). After addition of the extraction buffer, the cell material were shacked on a rocker in an ice bath for 80 minutes. The supernatant was obtained after centrifugation at 18000 rpm for 20 minutes to yield the “extract” for testing.

An electrochemiluminescence assay is performed as follows:
Sequentially, to each well, extracts from MCF-7 or K562 cells were added (the amount of extract per well was varied from that extracted from 100 to 10,000 cells; control wells without extract were also used) and then 50 μl/well of a mixture of BIOTIN-498A and TAG-495A (at a concentration between 1 μg/ml each in the 50 μl added per well) diluted into the PBS assay buffer) were added to wells of a 96-well U-bottom polypropylene plate and are incubated at room temperature with constant shaking for 2 hours.
10 μg of magnetic streptavidin beads (e.g., Dynabeads M-280 Streptavidin, Catalog #110028, BioVeris Corporation, Gaithersburg, Md.) in 25 μl were added to each well and incubated with constant shaking for 30 minutes.
PBS assay buffer was added to each well to make a final volume of 250 μl per well. All conditions were tested in at least quadruplicate wells. The 96 well plate was then analyzed for electrochemiluminescence using the M-Series® 384 Analyzer (BioVeris, Corporation, Gaithersburg, Md.).

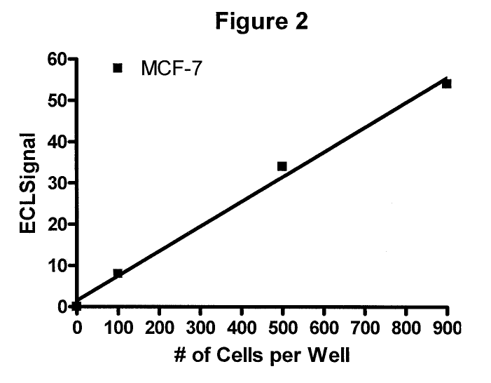

Using this immunoassay, ER-alpha was detectable from small numbers of MCF-7 cells per well and ECL signals from K562 extracts were lower than from MCF-7 cells (Table 5 and FIGS. 2 & 3), indicating the high sensitivity and high specificity of the immunoassay.

TABLE 5
Comparison of the ECL signal for the immunoassay detection
of ER-alpha in MCF-7 cells (positive control for high levels
of ER-alpha) vs. K562 cells (negative control).

Mean ECL Signal (above background) ±
Standard Error of the Mean

| Cells/well | MCF-7 | K562 |
|---|---|---|
| 0 | 0 ± 2 | 0 ± 2 |
| 100 | 8 ± 2 | 6 ± 6 |
| 500 | 34 ± 5 | 6 ± 4 |
| 900 | 54 ± 4 | 14 ± 5 |
| 10,000 | 432 ± 10 | 11 ± 5 |

Wells with no cells were run 8 times; all other samples were run in quadruplicate.

1. A method of detecting the expression of a steroid receptor from circulating carcinoma cells in a blood sample comprising isolating the carcinoma cells from the blood sample followed by making an extract from the isolated carcinoma cells followed by performing on the extract an immunoassay capable of detecting said steroid receptor, in which a positive immunoassay result indicates the presence of the steroid receptor in the carcinoma cells;
wherein the carcinoma cells are breast cancer cells and the steroid receptor is selected from the group consisting of estrogen receptor-alpha, estrogen receptor-beta, progesterone receptor, and androgen receptor, or the carcinoma cells are prostate cancer cells and the steroid receptor is androgen receptor;
wherein the immunoassay has a sensitivity defined by being capable of detecting the steroid receptor from nine hundred MCF-7 carcinoma cells which are spiked into a milliliter of a blood sample from a person without carcinoma, or by being capable of detecting 4 pg of the steroid receptor.

2. The method of claim 1, wherein the immunoassay is capable of detecting the steroid receptor from five hundred MCF-7 carcinoma cells which are spiked into a milliliter of a blood sample from a person without carcinoma.

3. The method of claim 2, wherein the immunoassay is capable of detecting the steroid receptor from one hundred MCF-7 carcinoma cells which are spiked into a milliliter of a blood sample from a person without breast carcinoma.

4. The method of claim 3, wherein the immunoassay is capable of detecting the steroid receptor from thirty MCF-7 carcinoma cells spiked into a milliliter of a blood sample from a person without breast carcinoma.

5. The method of claim 1, wherein the immunoassay uses electrochemiluminescence for detection.

6. The method of claim 1, wherein the immunoassay uses a technique selected from chemiluminescence, fluorogenic chemiluminescence, fluorescence polarization, and time-resolved fluorescence for detection.

7. The method of claim 1, wherein the immunoassay uses a polyclonal antibody against the steroid receptor.

8. The method of claim 1, wherein the immunoassay uses a first polyclonal antibody against the N-terminal region of the steroid receptor and a second polyclonal antibody against the C-terminal region of the steroid receptor.

9. The method of claim 1, wherein the immunoassay uses a monoclonal antibody against the steroid receptor.

10. The method of claim 1, wherein the carcinoma cells are isolated by contacting the blood with immunomagnetic beads capable of binding selectively to the carcinoma cells.

11. A method of claim 1 wherein the steroid receptor is estrogen receptor-alpha.

12. A method of claim 1 wherein the steroid receptor is progesterone receptor.

13. A method of identifying a cancer patient likely to benefit from treatment with an endocrine therapeutic agent, comprising the method of claim 1, wherein the carcinoma cell-containing blood sample is drawn from the patient.

14. The method of claim 13, wherein tumor tissue from the patient has been previously determined immunohistochemically to be negative for expression of the steroid receptor.

15. A method of treating a cancer patient likely to benefit from treatment with an endocrine therapeutic agent, comprising administering the anticancer agent to the patient identified according to the method of claim 13, thereby treating the patient.

16. The method of claim 15, wherein the endocrine therapeutic agent is selected from the group consisting of tamoxifen, an aromatase inhibitor, and fulvestrant.

17. The method of claim 16, wherein the aromatase inhibitor is selected from the group consisting of anastrozole, letrozole, exemestane, and fadrozole.
